(19) United States
(12) Patent Application Publication
(10) Pub. No.: US 2005/0038077 A1
(43) Pub. Date: Feb. 17, 2005

(54) Tablet containing 3-[(2-{[4-(hexyloxycarbonylamino-imino-methyl)-phenylamino]-methyl}-1-methyl-1H- benzimidazol-5-carbonyl)-pyridin-2-yl-amino]-propionic acid ethylester or the salts thereof

(75) Inventor: Anja Kohlrausch, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/901,809

(22) Filed: Jul. 29, 2004

Related U.S. Application Data
(60) Provisional application No. 60/500,753, filed on Sep. 5, 2003.

(30) Foreign Application Priority Data
Aug. 16, 2003 (DE) DE 103 37 697

(51) Int. Cl. A61K 31/4439; A61K 9/20

(52) U.S. Cl. 514/338; 424/464

(57) ABSTRACT

A tablet comprising: (a) ethyl 3-[(2-{[4-(hexyloxycarbonylaminoiminomethyl)phenylamino]methyl}-1-methyl-1H-benzimidazole-5-carbonyl)pyridin-2-ylamino]propionate or a pharmaceutically acceptable salt thereof; (b) one or more pharmaceutically acceptable organic acids with a solubility in water of >1 g/250 mL at 20° C.; and (c) a pharmaceutically acceptable excipient or filler.

### RELATED APPLICATIONS

This application claims benefit of U.S. Ser. No. 60/500,753, filed Sep. 5, 2003, and claims priority to German Application No. 103 37 697.6, filed Aug. 16, 2003, each of which is hereby incorporated by reference in its entirety.

### FIELD OF THE INVENTION

The invention relates to a tablet for the active substance ethyl 3-[(2-{[4-(hexyloxycarbonylaminoiminomethyl)phenylamino]methyl}-1-methyl-1H-benzimidazole-5-carbonyl)pyridin-2-ylamino]propionate or the pharmacologically acceptable salts thereof, which has the chemical formula (I) below.

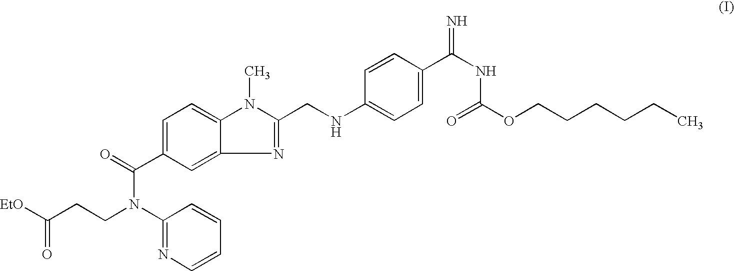

The compound of formula (I) is already known from WO 98/37075 (corresponding to U.S. Pat. Nos. 6,087,380; 6,469,039; 6,414,008; and 6,710,055, which are each hereby incorporated by reference), in which compounds with a thrombin-inhibiting and thrombin time-prolonging activity are disclosed, under the name 1-methyl-2-[N-[4-(N-n-hexyloxycarbonylamidino)phenyl]aminomethyl]benzimidazol-5-ylcarboxylic acid-N-(2-pyridyl)-N-(2-ethoxycarbonylethyl)amides. The compound of formula (I) is a double prodrug of the compound of formula (II) 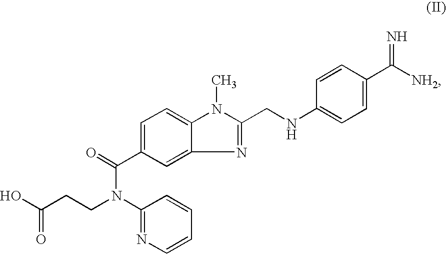
i.e., the compound of formula (I) is only converted into the compound which is actually effective, namely the compound of formula (II), in the body. The main range of indications for the compound of chemical formula (I) is the postoperative prophylaxis of deep vein thrombosis.

### DESCRIPTION OF THE INVENTION

The aim of the invention is to provide an improved formulation for oral use for the compound of formula (I), which is also referred to hereinafter as the active substance.

Surprisingly it has now been found that the use of pharmaceutically acceptable organic acids with a solubility in water of >1 g/250 mL at 20° C., preferably >1 g/160 mL at 25° C., in solid oral formulations leads to a significantly improved galenic form of ethyl 3-[(2-{[4-(hexyloxycarbonylaminoiminomethyl)phenylamino]methyl}-1-methyl-1H-benzimidazole-5-carbonyl)pyridin-2-ylamino]propionate and the pharmaceutically acceptable salts thereof.

Pharmaceutically suitable acids for the purposes of this invention are, for example, tartaric acid, fumaric acid, succinic acid, citric acid, and malic acid including the hydrates and acid salts thereof. Fumaric acid is particularly suitable for the purposes of this invention.

A preferred embodiment of the invention is a tablet.

The tablets contain 5 wt. % to 50 wt. % of active substance (based on the methanesulfonate), 5 wt. % to 50 wt. % of a pharmaceutically acceptable organic acid with a solubility in water of >1 g/250 mL at 20° C. as well as other excipients and fillers. Examples of other excipients and fillers which may be used include, for example, 1 wt. % to 80 wt. % of a filler, optionally up to 10 wt. % of a binder (i.e., 0 wt. % to 10 wt. % of binder), 1 wt. % to 10 wt. % of a disintegration promoter and 0.25 wt. % to 10 wt. % of a lubricant, with all the ingredients adding up to 100 wt. %.

Tablets which contain 10 wt. % to 30 wt. % active substance (based on the methanesulfonate), 10 wt. % to 40 wt. % of a pharmaceutically acceptable organic acid, 5 wt. % to 70 wt. % of a filler, 3 wt. % to 5 wt. % of a binder, 2 wt. % to 6 wt. % of a disintegration promoter, and 1 wt. % to 5 wt. % of a lubricant are preferred.

Particularly preferred are tablets which contain 15 wt. % to 25 wt. % of active substance (based on the methanesulfonate), 10 wt. % to 30 wt. % of a pharmaceutically acceptable organic acid, 50 wt. % to 65 wt. % of a filler, 3 wt. % to 5 wt. % of a disintegration promoter, and 1.5 wt. % to 2.5 wt. % of a lubricant.

The acid ingredient used may a pharmaceutically acceptable organic acid with a solubility in water of >1 g/250 mL at 20° C., such as, e.g., tartaric acid, fumaric acid, succinic acid, citric acid, and malic acid, including the hydrates and acid salts thereof. The pharmaceutically acceptable organic acids used are preferably tartaric acid, fumaric acid, succinic acid, or citric acid; fumaric acid is particularly preferred.

By the active substance is meant the compound of formula (I) or one of the pharmaceutically acceptable salts thereof. The methanesulfonate (mesylate) of the compound of formula (I) is preferred.

The fillers, binders, disintegration promoters, and lubricants mentioned above are known compounds having the specified properties conventionally used in the pharmaceutical industry.

Preferred fillers which may be used are mannitol, erythritol, lactose, microcrystalline cellulose, hydroxypropylcellulose, particularly low-substituted hydroxypropylcellulose, and pregelatinized starch. It is particularly preferable to use mannitol.

The binder used may preferably be a partially or totally synthetic selected from among the polyvinylpyrrolidones (povidone) or copolymers of N-vinylpyrrolidone and vinyl acetate (copovidone) or hydroxypropylmethylcellulose.

Examples of preferred disintegration promoters include cross-linked polyvinylpyrrolidone (crospovidone), sodium starch glycolate, or cross-linked cellulosecarboxymethylether sodium salt (croscarmellose sodium). Crospovidone is particularly preferred.

Preferred lubricants include, for example, magnesium stearate, sodium stearyl fumarate, and saccharose fatty acid esters. Magnesium stearate is particularly preferred.

The tablets may be prepared by the methods described below:

Preparation of the Tablets

The tablet according to the invention may be prepared by directly mixing and compressing the ingredients or by dry granulation and compression. To prepare the tablet according to the invention, the following procedure may be used, for example.

The active substance, the acid, and a filler, e.g., mannitol, are premixed in an intensive mixer and then screened. The powder mixture is transferred into a gravity mixer, a disintegration promoter, e.g., crospovidone and optionally other excipients (e.g., a binder, if necessary) are added and then mixed together. After the addition of lubricants, particularly magnesium stearate and saccharose fatty acid esters, the ingredients are mixed again. The mixture of active substance and excipient thus obtained is then compressed using a suitable tablet press to produce the tablets according to the invention.

The content of active substance in the pharmaceutical composition is 5% to 50%, preferably 10% to 30%; the content of pharmaceutically acceptable organic acid is usually between 5% and 50%, preferably between 10% and 40%.

Unless otherwise stated, the percentages given are percent by weight in each case.

In the first clinical trials with conventional tablets containing the compound of formula (I), it had been found that highly variable plasma levels occurred, ranging to individual cases of malabsorption. The variability of the plasma level patterns is significantly lower when the compound of formula (I) is administered as an oral solution; no instances of malabsorption have been observed.

One advantage of the formulation according to the invention containing the compound of formula (I) is that it guarantees sufficient bioavailability of the active substance which is better than that obtained with a conventional pharmaceutical preparation and is largely independent of the pH of the stomach, it reduces fluctuations in the bioavailability of the active substance and prevents malabsorption. Another advantageous property of the pharmaceutical composition according to the invention is its suitability for all patients, i.e., including those whose gastric pH is raised as a result of normal physiological variability, illness, or co-medication with drugs which increase the gastric pH (e.g., pantoprazole).

The dosage for oral administration is conveniently 25 mg to 300 mg of the active substance base (per tablet), preferably 50 mg to 200 mg, particularly preferably 75 mg to 150 mg of the active substance base, once or twice a day in each case.

The Examples that follow are intended to illustrate the invention:

EXAMPLE 1
Tablets Containing 50 mg of Active Substance

| Ingredients | Ingredients | Ingredients |
|---|---|---|
| mesylate of the compound of formula (I)* | 57.655 | 16.957 |
| mannitol | 205.145 | 60.337 |
| fumaric acid | 50.000 | 14.706 |
| crospovidone | 13.600 | 4.000 |
| saccharose fatty acid ester | 6.800 | 2.000 |
| magnesium stearate | 6.800 | 2.000 |
| total | 340.000 | 100.000 |

*corresponds to 50 mg of the compound of formula (I)

EXAMPLE 2
Tablets Containing 100 mg of Active Substance

| Ingredients | mg/tablet | %/tablet |
|---|---|---|
| mesylate of the compound of formula (I)* | 115.310 | 16.957 |
| mannitol | 410.290 | 60.337 |
| fumaric acid | 100.000 | 14.706 |
| crospovidone | 27.200 | 4.000 |
| saccharose fatty acid ester | 13.600 | 2.000 |
| magnesium stearate | 13.600 | 2.000 |
| total | 680.000 | 100.000 |

*corresponds to 150 mg of the compound of formula (I)

EXAMPLE 3
Tablets Containing 150 mg of Active Substance

| Ingredients | mg/tablet | %/tablet |
|---|---|---|
| mesylate of the compound of formula (I)* | 172.963 | 23.062 |
| mannitol | 382.037 | 50.938 |
| fumaric acid | 150.000 | 20.000 |
| crospovidone | 30.000 | 4.000 |
| sodium stearyl fumarate | 15.000 | 2.000 |
| total | 750.000 | 100.000 |

*corresponds to 150 mg of the compound of formula (I)

### EXAMPLE 4

### Preparation of ethyl 3-[(2-{[4-(hexyloxycarbonylaminoiminomethyl)phenylamino]methyl}-1-methyl-1H-benzimidazole-5-carbonyl)pyridin-2-ylamino]propionate methanesulfonate

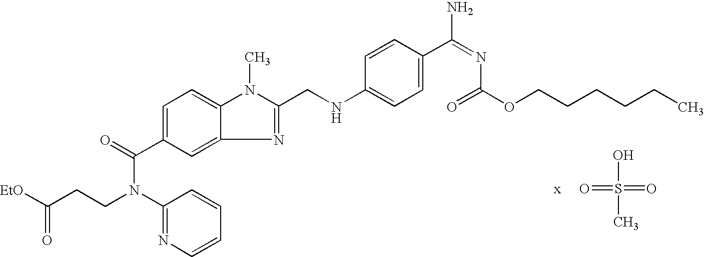

A solution of 5.0 mmol of methanesulfonic acid in 25 mL of ethyl acetate was added dropwise, with stirring, at ambient room temperature, to a solution of 3139 mg (5.0 mmol) of ethyl 3-[(2-{[4-(hexyloxycarbonylaminoiminomethyl)phenylamino]methyl}-1-methyl-1H-benzimidazole-5-carbonyl)pyridin-2-yl-amino]propionate base (prepared as described in WO 98/37075), in 250 mL of ethyl acetate. After a few minutes, the product started to crystallize out. It was stirred for one hour at ambient room temperature and for a further hour while cooling with ice, then the precipitate was suction filtered, washed with approximately 50 mL of ethyl acetate and 50 mL diethyl ether, and dried at 50° C. in the circulating air dryer. Yield: 94% of theory; pelting point: 178° C.-179° C.; C₃₄H₄₁N₇O₅ x

1. A tablet comprising:
(a) ethyl 3-[(2-{[4-(hexyloxycarbonylaminoiminomethyl)phenylamino]methyl}-1-methyl-1H-benzimidazole-5-carbonyl)pyridin-2-ylamino]propionate or a pharmaceutically acceptable salt thereof;
(b) one or more pharmaceutically acceptable organic acids with a solubility in water of >1 g/250 mL at 20° C.; and
(c) a pharmaceutically acceptable excipient or filler.

2. The tablet according to claim 1, wherein the pharmaceutically acceptable organic acid is tartaric acid, fumaric acid, succinic acid, citric acid, or malic acid, a hydrate or acid salt thereof.

3. The tablet according to claim 2, wherein the pharmaceutically acceptable organic acid is fumaric acid.

4. The tablet according to claim 1, wherein the amount of the ethyl 3-[(2-{[4-(hexyloxycarbonylaminoiminomethyl)phenylamino]methyl}-1H-methyl-1H-benzimidazole-5-carbonyl)pyridin-2-ylamino]propionate or the salt thereof is 5% to 50% of the tablet, based on the methanesulfonate.

5. The tablet according to claim 2, wherein the amount of the ethyl 3-[(2-{[4-(hexyloxycarbonylaminoiminomethyl)phenylamino]methyl}-1-methyl-1H-benzimidazole-5-carbonyl)pyridin-2-ylamino]propionate or the salt thereof is 5% to 50% of the tablet, based on the methanesulfonate.

6. The tablet according to claim 3, wherein the amount of the ethyl 3-[(2-{[4-(hexyloxycarbonylaminoiminomethyl)phenylamino]methyl}-1-methyl-1H-benzimidazole-5-carbonyl)pyridin-2-ylamino]propionate or the salt thereof is 5% to 50% of the tablet, based on the methanesulfonate.

7. The tablet according to claim 1, wherein the amount of the pharmaceutically acceptable organic acid is 5% to 50% of the tablet.

8. The tablet according to claim 2, wherein the amount of the pharmaceutically acceptable organic acid is 5% to 50% of the tablet.

9. The tablet according to claim 3, wherein the amount of the pharmaceutically acceptable organic acid is 5% to 50% of the tablet.

10. The tablet according to claim 4, wherein the amount of the pharmaceutically acceptable organic acid is 5% to 50% of the tablet.

11. The tablet according to claim 5, wherein the amount of the pharmaceutically acceptable organic acid is 5% to 50% of the tablet.

12. The tablet according to claim 6, wherein the amount of the pharmaceutically acceptable organic acid is 5% to 50% of the tablet.
